(19) United States
(12) Patent Application Publication
(10) Pub. No.: US 2015/0366661 A1
(43) Pub. Date: Dec. 24, 2015

(54) DEVICE FOR FITTING AND REMOVING A PROTHESIS

(71) Applicant: PROTIP MEDICAL, Schiltigheim (FR)

(72) Inventor: Nicolas Perrin, Schiltigheim (FR)

(21) Appl. No.: 14/646,356

(22) PCT Filed: Nov. 20, 2013
(86) PCT No.: PCT/EP2013/074322

§ 371 Date: May 20, 2015

(30) Foreign Application Priority Data
Nov. 20, 2012 (FR) 12/61029

(51) Int. Cl. A61F 2/20

(52) U.S. Cl. CPC A61F 2/20; A61F 2002/046

(57) ABSTRACT

The invention relates to a device (1) for fitting and removing a prosthesis (40), comprising:
attachment means (2) attachable to the prosthesis (40) and capable of being arranged in two positions,
   a closed position, in which said attachment means are secured to the prosthesis (40), and
   an open position, in which said attachment means release the prosthesis;
grasping means (3);
control means (4) connected to the attachment means (2), the control means (4) being capable of controlling the attachment means (2) such as to place same in the closed position or the open position;
at least partially flexible connecting means (5) that connect the control means (4) to the attachment means (2).

### TECHNICAL FIELD

The present invention relates to a device for fitting and removing a prosthesis, which is preferably an intralaryngeal prosthesis, and to a kit for fitting and removing a prosthesis, which is likewise preferably an intralaryngeal prosthesis.

### PRIOR ART

French patent application no. 11 02694 describes an intralaryngeal prosthesis which has excellent resistance to wear and to deformation under physiological conditions and which also has a long durability (lifespan). This intralaryngeal prosthesis is made of solid biocompatible metal and is intended to be implanted within a dysfunctional larynx, said device having a distal portion that forms an annular bearing structure and a central portion that forms a shutter intended to allow air to pass through and to hermetically prevent any other element from passing through, the shutter comprising i) a peripheral part forming a first valve flap rigidly connected to the annular bearing structure at a first hinge region, and ii) a central part forming a second valve flap rigidly connected to the first valve flap at a second hinge region, said first and second valve flaps cooperating with each other in a completely hermetic manner.

To date, this intralaryngeal prosthesis has been placed in the larynx of the patient manually by the surgeon. However, this manual placement was complicated, lengthy and somewhat imprecise. Moreover, it was complicated or even impossible to place this prosthesis in patients who had not undergone a tracheotomy. This is because the operating method for fitting the intralaryngeal prosthesis required the surgeon to position the prosthesis correctly by “pulling” it through the tracheostomy. The placement of the prosthesis in patients with a partially dysfunctional larynx (such as patients who have problems swallowing) thus required a tracheotomy to be performed before correcting the problem, which resulted in the patient suffering another secondary effect.

It is therefore desirable to prevent and treat dysfunctions of the larynx by allowing a prosthesis to be fitted which is able to restore normal functioning of the larynx without the need to perform a tracheotomy on the patient. The applicant has therefore developed an ancillary for fitting and removing such prostheses, which ancillary can be operated directly and uniquely by the oral route.

WO 96/35399 describes a voice prosthesis and also an ancillary for implanting same. The access route is through the tracheostomy (FIG. 1). This device uses the rotation of the elements relative to one another and has an axis of revolution. The connecting means are not flexible, since they have to permit the deformation of part of the prosthesis for implantation.

WO 2011/051177 also describes an ancillary for implanting a voice prosthesis. The access route is through the tracheostomy (FIG. 19). This device uses the rotation of the elements relative to one another and has an axis of revolution. The connecting means do not appear to be flexible.

WO 2005/097001 describes an ancillary for implanting a prosthesis through a flexible wall, which is accessible only from one side. The access route is through the tracheostomy present after the total laryngectomy (page 3, lines 15-19). On account of the operating method (it is necessary to push on the ancillary (see FIGS. 14 through 16)), the device does not have a flexible part.

These three devices also require the operating surgeon to use both hands.

U.S. Pat. No. 6,666,208 describes an ancillary for implanting a voice prosthesis. The access route is through the tracheostomy (column 3, lines 1-2). This device does not use connecting means that are at least partly flexible.

U.S. Pat. No. 6,565,581 describes a device for performing an anastomosis. The device for fitting stents does not have connecting means that are at least partly flexible.

### DISCLOSURE OF THE INVENTION

The invention aims to overcome the disadvantages of the prior art by making available a device with which a prosthesis, in particular an intralaryngeal prosthesis, can be easily fitted in place in the body of a patient. In particular, this device permits access by the oral route and requires the operating surgeon to use just one hand.

To this end, a first aspect of the invention concerns a device for fitting and removing a prosthesis, said device comprising:
attachment means that can be attached to the prosthesis, the attachment means being able to be arranged in two positions:
   a closed position, in which they are rigidly connected to the prosthesis;
   an open position, in which they are not rigidly connected to the prosthesis;
gripping means for manipulation of the fitting and removing device;
control means, the control means being connected to the attachment means, the control means being able to control the attachment means in such a way as to place same in the closed position or the open position;
at least partially flexible connecting means that connect the control means to the attachment means in such a way as to transmit the command from the control means to the attachment means.

The gripping means are intended to remain outside the patient and to allow the surgeon, from outside the body of the patient, to manipulate the prosthesis that will be inserted into said body of the patient. The connecting means will therefore have a length that allows the gripping means to remain outside the body of the patient while the attachment means are situated in the body of the patient, at the location where the prosthesis is to be fitted. The connecting means will therefore be intended to have one end outside the patient, while the other end will be in the body of the patient.

The attachment means have a geometry and dimensions that are adapted to the prosthesis to which they are intended to be attached.

When the fitting and removing device is intended to fit and remove an intralaryngeal prosthesis, the attachment means are preferably able to be inserted into a larynx by the oral route. For this purpose, their external dimensions are preferably smaller than those of the conduits in which they are intended to be inserted.

These attachment means must necessarily be rigid in order to ensure their main function of correct positioning and attachment of the prosthesis.

A material is therefore used in particular that has excellent mechanical, electrical and wear properties, and also good stability. Preferably, this material also has a low coefficient of friction for uses in wet and dry environments. The material used is in particular Pomalux® (acetal copolymer from Westlake Plastics, Lenni, Pa., United States).

The attachment means must have a small size in order in particular to pass between the teeth of the patient: the external diameter of the largest component is therefore preferably less than 25 mm, of the order of 24.30 mm+/−0.1.

When the attachment means are in the closed position, they capture the prosthesis, thus allowing the latter to be moved to the desired location in the body of the patient. Once the prosthesis is in the desired position, the attachment means can be placed in the open position, in which they release the prosthesis. The fitting and removing device can then be withdrawn from the body of the patient, while the prosthesis remains in place. The movement of the attachment means from the closed position to the open position, and vice versa, is controlled from outside the body of the patient by the control means.

The attachment means make it possible to secure the fitting of the prosthesis, since the attachment means are arranged in such a way that the prosthesis cannot detach from the fitting and removing device as long as the attachment means are in the closed position.

Furthermore, the attachment means are preferably configured in such a way that the prosthesis can be inserted into the attachment means when and only when the attachment means are in the open position.

The device according to the invention thus allows a prosthesis to be fitted easily and safely.

Moreover, it can also be used to remove a prosthesis. To this end, the fitting and removing device is introduced into the body of the patient until it reaches the prosthesis. The attachment means are placed in the open position. Then, when the attachment means are in the desired position with respect to the prosthesis, they are placed in the closed position in such a way that they connect rigidly to the prosthesis. The attachment means are then withdrawn from the body of the patient and they carry the prosthesis with them. The fitting and removing device according to the invention thus allows a prosthesis to be withdrawn easily from the body of a patient.

The gripping means preferably have a handle.

The control means are preferably arranged on the gripping means in order to facilitate the use of the fitting and removing device by a surgeon. This is because the surgeon can then hold the gripping means in one hand and can control the position of the attachment means via the control means using the same hand, for example using the thumb of the same hand.

According to a preferred embodiment, the attachment means have:
an assembly zone that can be joined to a part of the prosthesis;
an elastically deformable tooth provided with retention means that are able to be retained by a complementary part of the prosthesis, the tooth having to deform elastically in at least one direction called the “release direction” in order to allow the retention means to free themselves from the complementary part of the prosthesis;
blocking means that can be placed in two positions:
   a closed position, in which they prevent the deformation of the tooth in the release direction;
   an open position, in which they do not prevent the deformation of the tooth in the release direction.

The assembly zone allows the prosthesis to be joined to the fitting and removing device. However, in the absence of the tooth and of the retention means, the prosthesis can easily detach from the fitting and removing device. The tooth provided with the retention means allows the prosthesis to be connected to the fitting and removing device. This connection can be undone when the blocking means are in the open position. By contrast, as long as the blocking means are in the closed position, the prosthesis cannot detach from the fitting and removing device. The attachment means thus formed are easy to control and are very reliable.

According to a first preferred embodiment, the retention means have a protrusion that can be received in the complementary part of the prosthesis, which part is thus formed by a seat of the prosthesis.

According to a second embodiment, the retention means could also be envisioned as having a cavity in which is engaged the complementary part of the prosthesis, formed by a protuberance of the prosthesis.

Advantageously, the blocking means are formed by a ring which is arranged in such a way as to prevent the movement of the tooth in the release direction when it is in the closed position.

According to different embodiments:
the attachment means can engage around a part of the prosthesis to which they are to be attached, or
the attachment means can engage inside a part of the prosthesis to which they are to be attached.

In the first case, the retention means preferably attach to an outer part of the prosthesis, and the ring is preferably configured in such a way that, when it is in the closed position, it surrounds a part of the prosthesis joined to the retention means. In this case, the assembly zone is preferably formed by an attachment seat that is able to receive one end of the prosthesis.

In the second case, the retention means preferably attach to an inner part of the prosthesis, and the ring is preferably configured in such a way that, when it is in the closed position, it is surrounded by a part of the prosthesis joined to the retention means. In this case, the assembly zone is preferably formed by a tenon that can insert itself into a mortise of the prosthesis.

The fitting and removing device preferably extends around a reference line which is at the center of the fitting and removing device. The reference line is preferably curved, since the fitting and removing device is preferably curved when at rest. Furthermore, the reference line follows the movements of the fitting and removing device when the latter deforms.

According to a preferred embodiment, the blocking means are able to move longitudinally so as to pass from the closed position to the open position, the control means having a cursor that can move longitudinally, the connecting means having a connecting tube which connects the cursor to the blocking means in such a way as to move the blocking means longitudinally when the cursor moves longitudinally. A fitting and removing device of this kind is easy to produce and is inexpensive and reliable. Moreover, a fitting and removing device of this kind is compact.

However, a fitting and removing device could also be envisioned in which the cursor and the blocking means can move in rotation around the reference line, or in a helical movement around the reference line, in order to pass from the closed position to the open position. In this case, the connecting means would preferably also have a connecting tube that connects the cursor to the blocking means in such a way as to move the blocking means in rotation or in a helical movement, respectively, when the cursor moves in rotation or in a helical movement.

Advantageously, the fitting and removing device is additionally provided with guide means in which a guide wire can be inserted. These guide means are able to facilitate the placement of the prosthesis in the body of the patient, and they permit a more precise positioning of the prosthesis. Moreover, they make it possible to stiffen the attachment means, so as to facilitate the insertion of the fitting and removing device into the body of the patient.

Advantageously, the guide means extend all the way through the fitting and removing device, which makes it easier to guide the fitting and removing device by sliding it on the guide wire that passes all the way through the fitting and removing device.

Moreover, the guide means are preferably arranged at the center of the fitting and removing device. Thus, the connecting tube preferably surrounds the guide tube. Likewise, the gripping means preferably surround the guide means, just as the attachment means preferably surround the guide means.

According to a preferred embodiment, the connecting means and the guide means form a flexible tube between the gripping means and the attachment means, which allows them to be compact and which facilitates the insertion of the fitting and removing device into the body of the patient (FIG. 11). The flexible tube is thus preferably formed by two concentric tubes, namely the connecting tube and the guide tube. The connecting tube can move in translation with respect to the guide tube in order to move the blocking means in translation. The connecting tube can thus slide with respect to the guide tube.

This flexible tube preferably has a length that is adapted depending on the location in the body where the prosthesis is to be placed.

According to a preferred embodiment, this flexible tube has a curvature at rest of between 130° and 180°, which makes it easier to insert the device into the body of the patient. The radius of curvature of the flexible tube is preferably 140 mm+/−20 mm at 30°+/−5°.

Thus, the ancillary preferably has a curvature permitting the following:
that the control elements have an ergonomic position for the user (the manipulation of the ancillary is not “impeded” by the face of the patient),
that the connecting elements follow the natural anatomical structure of the tongue (FIG. 11).

In this embodiment, the curvature (at rest) of the connecting means is defined by an angle of curvature of between 130° and 170°. Preferably, the ancillary described has a radius of curvature of 140 mm+/−20 mm at 30°+/−5°. It should be noted that the curved part of the connecting means is intended to be introduced into the body of the patient (in particular the oral orifice) and allows the prosthesis to pass beyond the base of the tongue during its positioning.

However, it is possible that the ancillary has an angle of curvature of 180° at rest. In this case, the connecting means and possibly the guide means are straight when at rest.

In this case, it is preferable that the ancillary has curve-inducing means by which it is possible to induce a curvature of the connecting means and guide means.

This result can be achieved in particular when the connecting means and possibly the guide means are in the form of rigidly connected segments which are immobile at rest and which are movable relative to one another when these curve-inducing means are activated. In this embodiment, there is therefore the possibility of deformation of the connecting means and possibly of the guide means if a force is applied to them.

The elements connecting the gripping means to the attachment means preferably have an external diameter of less than 11 mm, preferably of the order of 10.10 mm+/−0.1:
blocking tube (connecting tube): internal diameter 8.97 mm+/−0.1; external diameter 10.10 mm+/−0.1;
tooth guide tube (guide tube), guide element: internal diameter 6.97 mm+/−0.1; external diameter 8.10 mm+/−0.1;
guide wire: diameter 5.30 mm+/−0.2.

The fitting and removing device can also comprise locking means for keeping the blocking means in the closed position when they are in the closed position and/or for keeping the blocking means in the open position when they are in the open position, so as to avoid incorrect manipulations by the surgeon during the operation. These locking means are preferably formed by protruding parts that are positioned on the cursor and on the handle in such a way as to brake the passage of the cursor from the closed position to the open position, and vice versa. There is a “notch” to pass for each of the open and closed positions.

The fitting and removing device preferably permits the placement of an intralaryngeal prosthesis in the larynx of a patient. In this case, the flexible tube preferably has a length of between 10 and 15 cm, which allows the surgeon to manipulate the gripping means and control means while the attachment means are in the larynx of the patient. Moreover, the flexible tube must not be too long, so as to facilitate the insertion and the manipulation of the fitting and removing device, equipped at its end with the prosthesis, in the body of the patient.

Furthermore, the flexible tube must have a minimum stiffness so as to be able to push the attachment means, rigidly connected to the prosthesis, through the body of the patient.

Advantageously, the attachment means additionally have orientation means which allow the prosthesis and the assembly zone to be joined only when the prosthesis has a predetermined orientation relative to the assembly zone. These orientation means make it possible to control the orientation of the prosthesis in the fitting and removing device in order to know in which direction to insert the prosthesis in the fitting and removing device. These orientation means additionally make it possible to position the prosthesis in rotation in the larynx of the patient by turning the fitting and removing device around the reference axis. The orientation means thus make it possible to transmit a torque from the fitting and removing device to the prosthesis. If the prosthesis is not positioned correctly in rotation, the surgeon has to withdraw it from the larynx of the patient in order to reinsert it in the correct angular position. This is because the prosthesis, on its outer surface, can have protuberances that could injure the patient if the prosthesis was turned inside the body of the patient.

The orientation means are preferably arranged on a wall of the assembly zone. For example, when the assembly zone is an attachment seat into which the prosthesis is to be inserted, the orientation means are preferably formed by an opening in a wall of the attachment seat, into which opening a protuberance of the prosthesis can be inserted. This opening moreover allows the fitting and removing device to separate more easily from the prosthesis during the withdrawal of the fitting device by the surgeon.

The fitting and removing device is made of materials that are biocompatible at least in the short term. Thus, each of the connecting tube and guide tube is preferably made of polyethylene. The other components of the fitting and removing device are preferably made of polyoxymethylene or similar as seen above for the attachment means.

A second aspect of the invention likewise concerns a kit for fitting and removing a prosthesis, said kit having:
a fitting and removing device according to the first aspect of the invention;
a guide wire.

The guide wire is preferably made of PVC.

According to different embodiments, the guide wire can be straight or it can be curved, especially at at least one of its ends, in such a way as to optimize its guiding function in the body of the patient. The guide wire could in particular have an angled portion at one of its ends. This curved portion is preferably situated near an end of the guide.

This kit is preferably disposable. However, it would also be conceivable to be able to sterilize it several times, in which case it would be reusable.

In a preferred embodiment, said kit also has a prosthesis, preferably a laryngeal or intralaryngeal prosthesis, in particular of the kind described below.

Another aspect of the invention concerns a kit having a fitting and removing device, according to the first aspect of the invention, and also a laryngeal or intralaryngeal prosthesis, in particular of the kind described below.

The fitting and removing device is preferably a device for fitting and removing an intralaryngeal prosthesis.

The invention thus relates to this fitting and removing device for its implementation and its use for fitting and/or removing an intralaryngeal prosthesis by the oral route. In particular, this fitting and/or removal of the prosthesis is performed on a patient who has not undergone a tracheotomy. However, this fitting procedure can also be performed on patients who have undergone a tracheotomy. By using the ancillary according to the invention, it is then no longer necessary to “pull” the prosthesis through the tracheostomy.

Thus, the ancillary (and possibly the guide wire) preferably has a central lumen allowing air to pass from the outside to the airways and from the airways to the outside. Thus, the guide means and connecting means preferably have a central orifice.

The patient can thus breathe through the ancillary during the use of the ancillary.

A fourth aspect of the invention concerns a method for fitting a prosthesis, said method having a step of introducing the prosthesis into the body of a patient by the oral route with the aid of a fitting and removing device according to the invention.

The fitting method preferably has one or more of the following steps:
placing the attachment means of the fitting and removing device in the open position;
joining the prosthesis and the attachment means of the fitting and removing device;
placing the attachment means of the fitting and removing device in the closed position such that the attachment means and the prosthesis are rigidly connected;
inserting the guide wire through the fitting and removing device;
inserting the prosthesis into the body of the patient by the oral route with the aid of the fitting and removing device;
positioning the prosthesis in a final position; withdrawing the guide wire;
placing the attachment means of the fitting and removing device in the open position;
releasing the prosthesis by pulling the fitting and removing device back in the direction of the outside of the body of the patient, preferably with a small sharp movement;
withdrawing the fitting and removing device.

The inserted prosthesis is preferably an intralaryngeal prosthesis. It is preferably inserted by the oral route as far as the intralaryngeal space in which it is placed.

The fitting and removing device makes it possible to insert the prosthesis into the larynx of the patient by pushing it through the oral route, instead of inserting it by pulling it through a tracheostomy.

If the ancillary is used in such a way that the plane of symmetry of the ancillary is in the sagittal plane (median plane) of the patient, the angular positioning of the prosthesis will then be optimal (that is to say the plane of symmetry of the prosthesis will also be in the sagittal plane of the patient).

The pushing of the prosthesis through the throat of the patient can be monitored by endoscopy. The way of determining the correct positioning of the prosthesis, depending on the type of prosthesis used, will also be discussed.

A fifth aspect of the invention likewise concerns a method by which a prosthesis disposed in the body of a patient is removed by the oral route with the aid of a fitting and removing device according to the first aspect of the invention.

The method of removal preferably has one or more of the following steps:
placing the attachment means of the fitting and removing device in the open position;
inserting the fitting and removing device by the oral route until it reaches the prosthesis and the attachment means join to the prosthesis;
placing the attachment means in the closed position such that the prosthesis is rigidly connected to the attachment means;
withdrawing the prosthesis from the body of the patient with the aid of the fitting and removing device.

The prosthesis which is removed or withdrawn from the body of the patient is preferably an intralaryngeal or laryngeal prosthesis which was placed in the intralaryngeal space or as a replacement for the larynx of the patient.

In one embodiment, said laryngeal or intralaryngeal prosthesis can be as described in WO 2013/034858. This prosthesis can thus be a device with valve flaps, having a distal portion that forms an annular bearing structure and a central portion that forms a shutter intended to allow air to pass through and to hermetically prevent any other element from passing through, the shutter comprising i) a peripheral part forming a first valve flap rigidly connected to the annular bearing structure at a first hinge region, and ii) a central part forming a second valve flap rigidly connected to the first valve flap at a second hinge region (these valve flaps are described below). This device also comprises a means forming a collar or skirt situated below the annular bearing structure. In one specific embodiment, the annular bearing structure and the two valve flaps forming the shutter are made of solid metal (in particular titanium). This means forming a collar or skirt situated below the annular bearing structure is preferably made of silicone. Furthermore, this means forming a skirt is preferably composed of a first part situated at the top of the device and fixed to the annular bearing structure, a second part which has a smaller diameter than the first part and is situated below the latter, and finally a third part which has a diameter greater than that of the second part and is situated below the second part and comprises a bevel cut. It is preferable when the outer surface of the skirt is provided with lugs and/or when the inner surface of the skirt has a planar additional thickness over the entire height thereof, so as to increase the rigidity of the device. The lugs are intended to block the prosthesis in translation and rotation once it is in position within the larynx of the patient. Once in position, this prosthesis is blocked by the cricoid cartilage, in the area of the second part of the skirt (FIG. 11.B).

The internal diameter of the skirt preferably allows the passage of a 6.0 mm diameter cannula, so that medical examinations can be performed if required. Most of the sharp corners of the skirt (9) are rounded to make them non-traumatic, on the one hand, and to make removal of mucosities from the lower airways to the pharyngeal zone easier, on the other hand.

Another intralaryngeal prosthesis that can be used is described in EP 815807.

In another embodiment, said laryngeal or intralaryngeal prosthesis (FIG. 10) is advantageously composed of a body (81) made of biocompatible metal (preferably of solid titanium). In the upper part of this prosthesis (which will be situated proximally at the base of the tongue), there are two concentric valve flaps (82, 83). The opening of the small valve flap (83) in the downward direction permits inhalation, and the opening of both valve flaps (small+large) in the upward direction permits exhalation.

In one embodiment, the laryngeal or intralaryngeal prosthesis has a distal portion (81) which forms an annular bearing structure (and of which one part is intended to be fixed to a device that has been pre-positioned and fixed at the limit of the trachea of the patient, such as is described in WO 2013/079362), and a central portion that forms a shutter intended to allow air to pass through and to hermetically prevent any other element from passing through.

In a preferred embodiment, said shutter comprises i) a peripheral part forming a first valve flap (82) rigidly connected to the annular bearing structure at a first hinge region (84), said first valve flap (82) being able to lift under the effect of the raised pressure resulting from the exhalation by the patient, and ii) a central part forming a second valve flap (83) rigidly connected to the first valve flap (82) at a second hinge region (85), said second valve flap being able not only to drop under the effect of the reduced pressure exerted by the air inhaled by the patient but also to cooperate with the first valve flap in order to lift again following the exhalation by the patient, said first and second valve flaps cooperating with each other in a totally hermetic manner.

In a preferred embodiment, the orientation means present in the attachment means of the device according to the invention make it possible to orient the prosthesis with respect to the zone forming a hinge for the two valve flaps of said prosthesis, such as have been described above. This hinge zone in fact has to be applied against the base of the tongue of the patient, and it is therefore important that the surgeon knows the orientation of the prosthesis during its introduction, which is made possible by virtue of the presence of these orientation means.

It may also be noted that the hinges (84) and (85) are preferably made of a semi-rigid material or even a rigid material. The reason is that the movement must be made possible with a low respiratory pressure, but also the hinges have to allow the valve flaps to resist the pressure of the food bolus or of any fluid bearing with greater or lesser force on the shutter. Furthermore, it is necessary that the hinges resist colonization and ensure a regular and lasting movement of the valve flaps. Finally, the hinge zone has to ensure perfect stability of the axes of rotation with respect to the bearing structure of the device in order to guarantee the leaktightness upon closure.

In the preferred embodiment, a semi-rigid material of this kind (sufficiently flexible to be able to ensure a certain degree of elasticity, while at the same time being rigid enough to be able to resist a low pressure and retain its shape) is chosen from among plastic, gum, a resin or else silicone. A super-elastic material such as Nitinol can also be used. A preferred material is silicone, in particular silicone 70 Shore A.

The laryngeal or intralaryngeal prosthesis can also have an assistance device placed on the annular bearing structure (81) and/or the first valve flap (82). This assistance device can be mechanical, electrical or electronic, but it is preferably a magnetized device. Magnetized device or magnetized element is understood as one or more permanent magnets of the lanthanide type which are biocompatible or are rendered biocompatible by various treatments known to a person skilled in the art, or else are enclosed hermetically in a suitable housing provided for this purpose. Said magnetized device can be placed either on the first valve flap (82) or on the internal surface of the bearing structure (81). When the first valve flap is in the closed position, a metallic element is placed opposite the one or more magnets (or one or more metallic elements capable of being magnetized are placed in such a way as to come into contact with the one or more magnets when the first valve flap is in the closed position).

Thus, in one specific embodiment, the laryngeal or intralaryngeal prosthesis has an assistance device consisting of a metallic element arranged in the area of the annular bearing structure (81) and coming into contact with a magnetized element arranged in the area of the first valve flap (82). In a preferred embodiment, the laryngeal prosthesis has an assistance device consisting of a metallic element arranged in the area of the first valve flap (82) and coming into contact with a magnetized element arranged in the area of the annular bearing structure (81).

A mechanical assistance device can also be positioned on each of the valve flaps in order to facilitate their cooperation in the desired function. According to this embodiment, the laryngeal prosthesis is such that the first valve flap (82) and the second valve flap (83) comprise an assistance device so as to permit the lifting only of the second valve flap (83) and the simultaneous lowering of the first valve flap (82) and of the second valve flap (83). In a preferred embodiment, the valve flaps (82, 83) of the device according to the invention comprise an assistance device so as to permit the lowering only of the second valve flap (83) and the simultaneous lifting of the first valve flap (82) and of the second valve flap (83).

### BRIEF DESCRIPTION OF THE FIGURES

Other features and advantages of the invention will become clear from reading the following detailed description with reference to the attached figures, in which:

FIG. 1 shows a cross-sectional view of a fitting and removing device according to an embodiment of the invention;

FIG. 2 shows a cross-sectional view of the device from FIG. 1 into which an intralaryngeal prosthesis and a guide wire are inserted;

FIG. 3 shows a cross-sectional view of the attachment means of the fitting and removing device from FIG. 1 when they are in the open position and the intralaryngeal prosthesis has not yet been inserted into these attachment means;

FIG. 4 is a cross-sectional view of the attachment means from FIG. 3 when they are in the open position and the intralaryngeal prosthesis is inserted into the attachment means;

FIG. 5 shows a cross-sectional view of the attachment means from FIG. 4 when they are in the closed position on the intralaryngeal prosthesis;

FIG. 6 shows a perspective view of an insertion kit, having the device from FIG. 1, when it is in the open position;

FIG. 7 shows a perspective view of the device from FIG. 1 when it is in the open position and an intralaryngeal prosthesis has been inserted into this device;

FIG. 8 shows a perspective view of the device from FIG. 1 when it is in the closed position and an intralaryngeal prosthesis is attached to it;

FIG. 9 shows a cross-sectional view of the handle of the device from FIG. 1;

FIG. 10 shows a laryngeal prosthesis (81) which has the features needed to obtain a function similar to the natural larynx. This prosthesis is intended in particular to be fastened to a device of the kind described in WO 2013/079362;

FIG. 11 describes fitting an intralaryngeal prosthesis by the oral route. 11A: insertion of the prosthesis; 11B: positioning of the prosthesis. The deformation of the flexible connecting means of the device during the insertion of the prosthesis is therefore shown. This prosthesis is similar to the one described in WO 2013/034858.

For greater clarity, the features that are identical or similar are designated by identical reference signs in all of the figures.

### DETAILED DESCRIPTION OF AT LEAST ONE EMBODIMENT

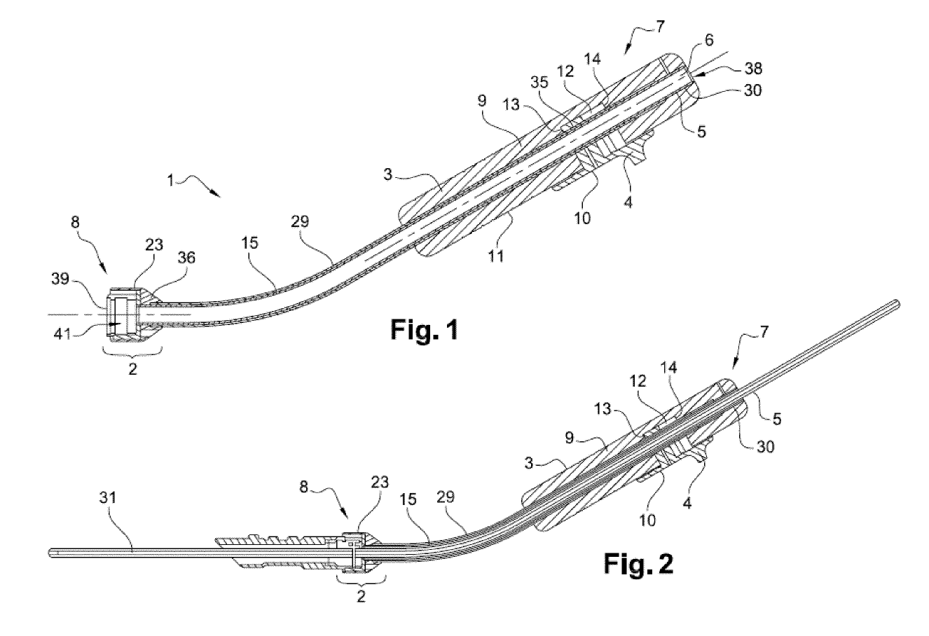

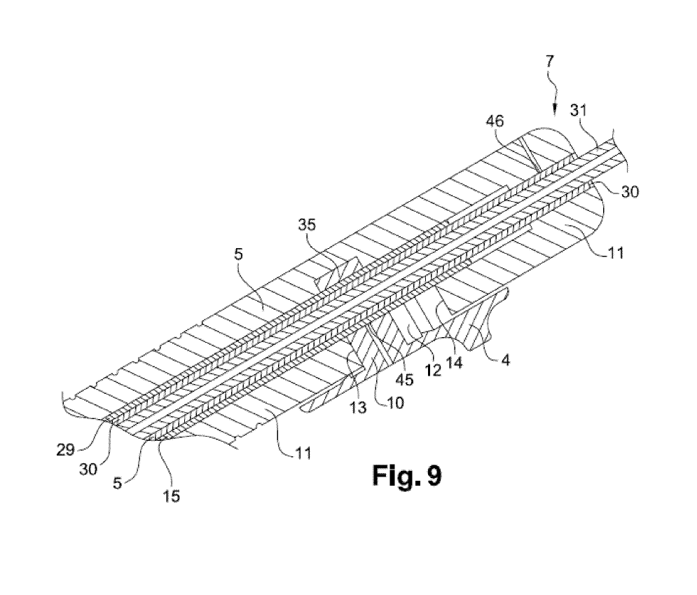

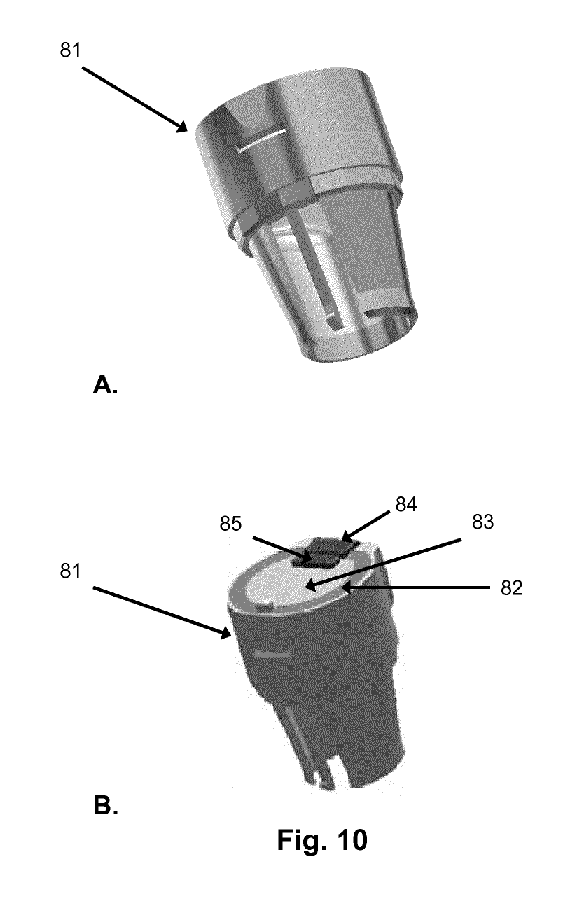

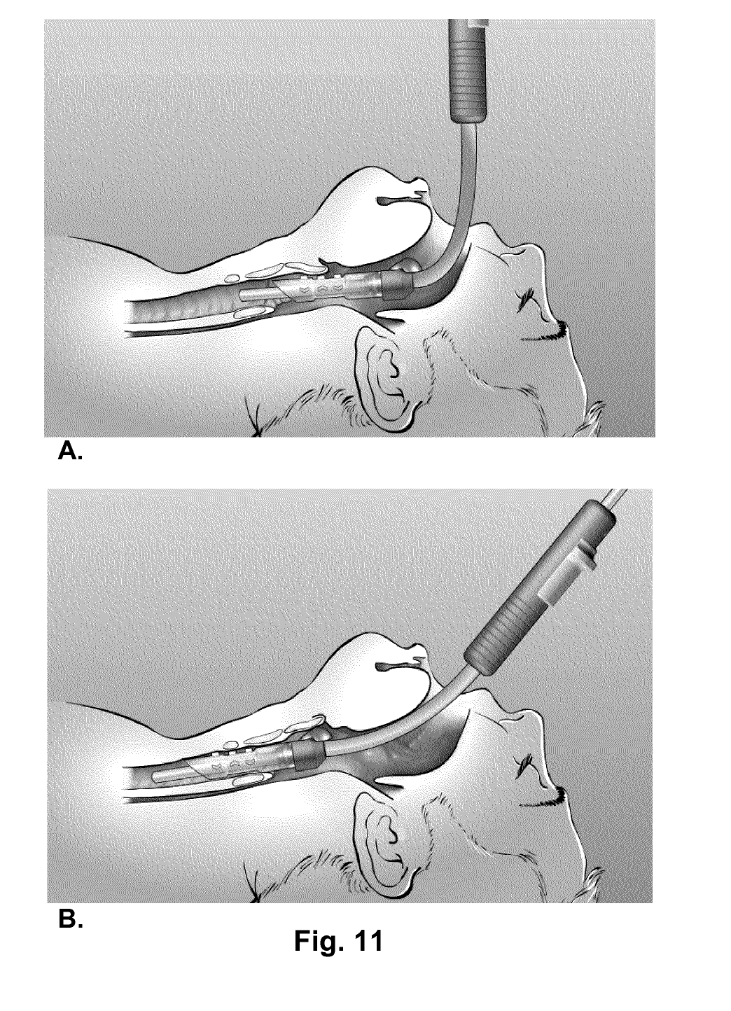

A fitting and removing device 1 according to an embodiment of the invention will now be described in more detail with reference to FIGS. 1 through 9. This fitting and removing device permits the placement of an intralaryngeal prosthesis 40. However, it could also permit the placement of other types of prostheses.

The fitting and removing device 1 extends along a reference line 6. This reference line 6 is curved since the device 1 has a curvature with a radius of curvature of preferably 140 mm+/−20 mm at 30°+/−5°. The curve of the reference line can be modified when the device is folded in one direction or the other. In this document, the term “longitudinal” or “axial” is used to describe an element that extends substantially along the reference line, and the term “transverse” describes an element that extends substantially perpendicularly with respect to the reference axis 6.

The fitting and removing device 1 has a proximal end 7 and a distal end 8. The fitting and removing device 1 has:
gripping means 3 situated at the proximal end 7;
control means 4 situated on the gripping means 3;
attachment means 2 situated at the distal end 8;
connecting means 5 which connect the control means 4 to the attachment means 2;
guide means 15.

In this illustrative embodiment, the gripping means 3 are formed by a handle 9 that extends along the longitudinal axis 6. The handle 9 in this example has a substantially cylindrical shape around the reference axis 6. The handle 9 is made of polyoxymethylene in this illustrative embodiment.

The control means 4 are formed by a cursor 10. The cursor 10 is situated on a lateral surface 11 of the handle 9. The cursor 10 is fitted in a seat 12 of the handle 9, in which the cursor 10 can be moved longitudinally between a closed position, in which it is in abutment against the transverse wall 13 of the seat 12 (case shown in FIG. 1), and an open position, in which it is in abutment against the transverse wall of the seat 12. The cursor 10 is made of polyoxymethylene in this example.

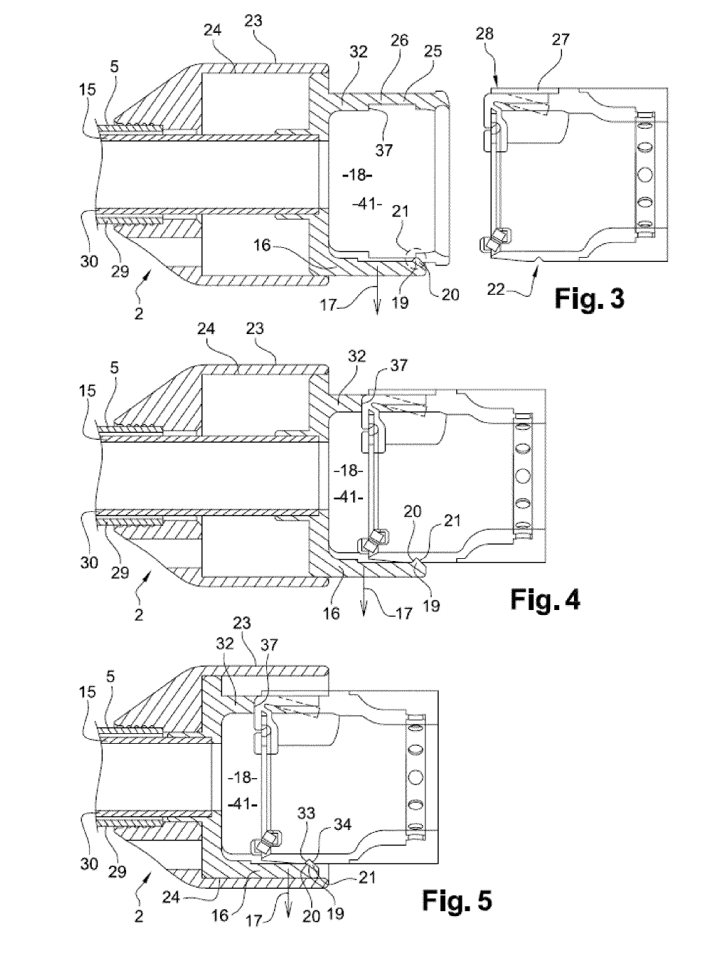

The attachment means 2 are shown in more detail in FIGS. 3 to 5. The attachment means 2 have an assembly zone 41 which is here formed by an attachment seat 18 into which an end 28 of a prosthesis can be introduced.

The attachment means 2 have orientation means 25 which allow the prosthesis to be introduced into the attachment seat 18 in a single position. In this embodiment, these orientation means 25 are formed by an opening 26 arranged on part of the circumference of the lateral wall 32 of the attachment seat 18. The prosthesis 40 has, at the end 28 thereof to be introduced into the attachment seat 18, a protuberance 27 dimensioned so as to be able to fit into the opening 26. The transverse dimensions of the attachment seat 18 are such that the end 28 of the prosthesis 40 cannot fit in the attachment seat 18 in any position other than that in which the protuberance 27 is fitted in the opening 26.

The attachment means 2 also have a tooth 16. The tooth is preferably formed on part of the circumference of the attachment seat 18. The tooth 16 is preferably positioned in such a way as to be diametrically opposite the opening 26. The tooth 16 protrudes axially on the lateral wall 32 of the attachment seat 18. The tooth 16 is designed in such a way as to be able to deform in a direction 17 called the “release direction”. The release direction 17 is preferably substantially perpendicular to the reference axis 6. For this, the attachment seat 18 and the tooth 16 are preferably made of polyoxymethylene. The tooth 16 can, for example, have a rectangular cross section, measuring 2.6 mm by 1.6 mm, and a length of 10 mm from the edge of the attachment seat 18.

The tooth 16 has a distal end 19 provided with retention means 20. These retention means 20 are here formed by a protrusion 21 which projects transversely from the tooth 16 in the direction of the interior of the attachment seat 18. The protrusion 21 is intended to be fitted in a seat 22 of the prosthesis 40. The protrusion 21 preferably has a triangular cross section formed by two walls 33, 34 inclined with respect to the reference axis and forming between them a rounded edge, which makes it easier for the protrusion 21 to leave the seat 22 of the prosthesis. By virtue of the presence of the inclined walls 33, 34, a protrusion 21 of this kind can easily enter and leave the seat 22 when the fitting and removing device moves in translation with respect to the prosthesis.

The attachment means 2 also have blocking means 23. The blocking means 23 are here formed by a ring 24 which can be moved in translation longitudinally along the reference axis 6 between a closed position, shown in FIG. 5, in which it extends along the tooth 16 as far as the distal end 19 of the latter, and an open position, shown in FIG. 3, in which it is set back from the assembly zone 41. The ring 24 has transverse dimensions which are such that, when the ring 24 is in the closed position, it extends along the full length of the tooth 16 so as to prevent the movements of the tooth 16 in the release direction 17.

The blocking means 23 are connected to the control means 4, more precisely to the cursor 10, by way of the connecting means 5. These connecting means 5 allow the blocking means 23 to be moved longitudinally in translation when the cursor 10 moves longitudinally in translation. More precisely, the connecting means 5 allow the ring 24 to be placed in the closed position when the cursor 10 is in the closed position, and they allow the ring 24 to be placed in the open position when the cursor 10 is in the open position. For this, the connecting means 5 preferably have a connecting tube 29 which has a proximal end 35 connected to the cursor 10 and a distal end 36 connected to the ring 24. The connecting tube 29 is preferably made of polyethylene. The proximal end 35 of the connecting tube 29 is preferably adhesively bonded to the cursor by virtue of at least one adhesive zone 45. Likewise, the end of the guide tube 30 is preferably attached to the handle 9 by an adhesive bonding zone 46. The distal end 36 of the connecting tube is preferably adhesively bonded to the ring 24.

The fitting and removing device 1 also has guide means 15 into which a guide wire can be inserted. The guide means 15 are preferably formed by a guide tube 30 into which a guide wire can be introduced. The guide tube 30 is preferably arranged in such a way as to be able to receive not only the guide wire but also an optical device making it possible to view the fitting of the prosthesis. The guide tube 30 is preferably made of polyethylene. This guide tube 30 preferably extends all the way through the fitting and removing device from the proximal end 7 thereof to the distal end 8 thereof. The guide tube 30 opens out in a transverse opening arranged in the area of the proximal end 7 of the fitting and removing device and called the “proximal aperture” 38. The guide tube 30 opens out in a transverse opening arranged in the area of the distal end and called the “distal aperture” 39. The guide tube 30 is preferably surrounded, between the gripping means 3 and the attachment means 2, by the connecting tube 29, such that in this portion the guide tube 30 and the connecting tube 29 form a flexible tube that is able to deform in order to adapt to the conduits into which it is introduced. However, this flexible tube must retain a minimum rigidity in order to be able to introduce the attachment means through the body of the patient.

It is therefore preferable that the materials used for the connecting means and for the guide wire have mechanical properties that permit:
their deformation in order to adapt to the different anatomies of the patients,
their mechanical strength in order to transmit the force needed for placement of the prosthesis (here in the larynx).

As regards the material used for the connecting elements, it is therefore especially suitable to use polyethylene, in particular combinations of high-density and low-density polyethylene (HDPE/LDPE).

The guide wire (31) must be more flexible, and use is made in particular of PVC.

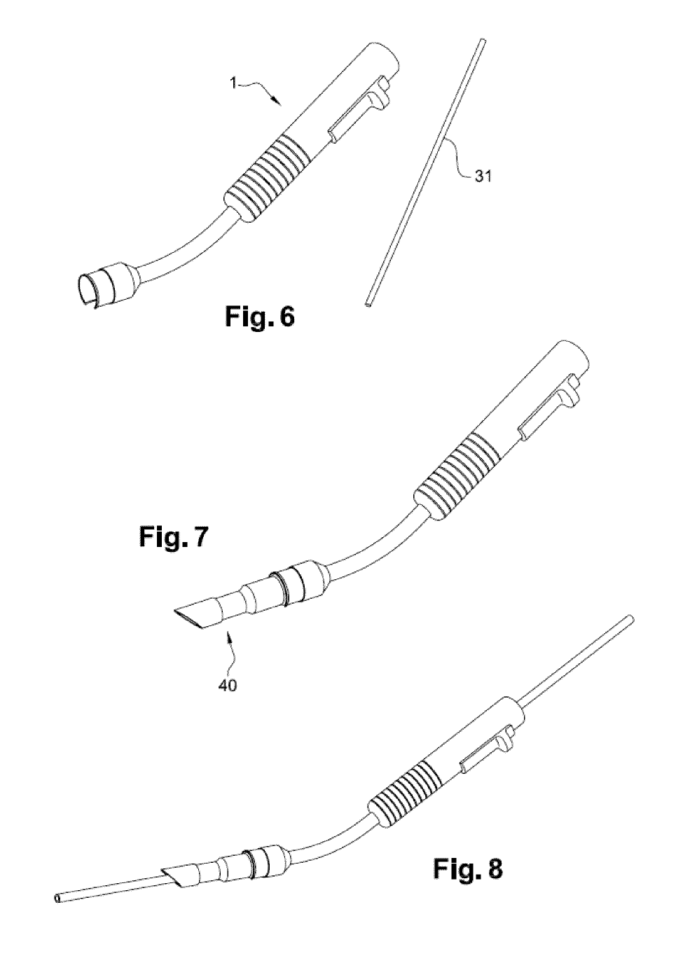

FIG. 6 shows a fitting and removing kit comprising the fitting and removing device, as described above, and also a guide wire 31. The guide wire 31 is preferably cylindrical. It is preferably made of polyethylene. It can have a diameter of 5.3 mm, for example, when the guide tube 30 has a diameter of 6.97 mm.

The function of the fitting and removing device, which has been described with reference to FIGS. 1 through 5, will now be described with reference to FIGS. 6 through 8.

In a first step, the attachment means are placed in the open position, as shown in FIG. 6. For this, the cursor 10 is moved in translation in the direction of the proximal end 7 of the fitting and removing device, such that it comes into abutment against the transverse wall 14. This translation of the cursor 10 brings about the translation of the blocking means 23 via the connecting means 5. The blocking means 23 are then in the open position shown in FIG. 5, in which position they do not prevent the movement of the tooth 16 in the release direction 17.

The prosthesis 40 is then inserted into the attachment means 2 and more specifically into the attachment seat 18, as is shown in FIG. 7, until the prosthesis comes into abutment against a limit stop 37 of the attachment seat 18. A single orientation of the prosthesis in the attachment seat 18 is possible by virtue of the orientation means 25. Indeed, the prosthesis can engage in the attachment seat 18 only when the protuberance 27 of the prosthesis faces toward the opening 26 of the fitting and removing device. Upon introduction of the end 28 of the prosthesis into the attachment seat 18, the retention means 20 of the tooth 16 engage in the seat 22 of the prosthesis following the deformation of the tooth 16 in the release direction 17. The end 28 of the prosthesis preferably has a ramp which makes it easier to introduce the prosthesis into the assembly zone despite the presence of the tooth provided with the retention means. This ramp makes it possible to initiate the deformation of the tooth in the release direction such that the end of the prosthesis engages in the attachment seat 18.

The cursor 10 is then moved in translation along the reference axis 6 in the direction of the distal end 8 of the fitting and removing device until it comes into abutment against the transverse wall 13. This translation of the cursor 10 brings about the translation of the blocking means 23 in the direction of the distal end 8 of the fitting and removing device via the connecting means 5. The blocking means 23 then change to the closed position since they extend along the tooth 16 in such a way as to prevent the movements of the latter in the release direction. The attachment means 2 are then in the closed position, such that they hold the prosthesis captive.

The guide wire 31 is then inserted into the guide tube 30 via the proximal aperture 38 until it emerges from the distal aperture 39 by approximately 10 cm, as is shown in FIG. 8.

With the aid of the fitting and removing device, the prosthesis is then pushed until it impacts the cricoid cartilage. The use of the guide wire 31 makes it possible to secure the path of the prosthesis and of its insertion system as far as the implantation site: it is thus possible, for example, to avoid taking the direction of the esophagus. The orientation means 25 allow the prosthesis to be positioned in rotation inside the larynx, since they make it possible to turn the prosthesis when the handle 9 is turned. The orientation of the prosthesis can thus be easily controlled from the outside by a surgeon using the handle 9. If the prosthesis is poorly positioned, it is easier to withdraw the prosthesis from the larynx and to reimpact it in the cricoid cartilage, instead of turning it directly within the body of the patient. It is thus possible to avoid a situation in which the tissues of the larynx are damaged by the silicone barbs of the prosthesis, the role of which barbs is precisely to prevent any rotation of the prosthesis once it has been implanted.

Once the prosthesis is in place, the guide wire 31 is withdrawn, and the attachment means are then once again placed in the open position. For this, the cursor 10 is moved in translation in the direction of the proximal end 7 of the fitting and removing device, which brings about the translation of the blocking means 23, which then no longer prevent the movement of the tooth 16 in the release direction 17. The prosthesis is then released from the fitting and removing device by applying a short sharp withdrawal movement of the fitting and removing device. The prosthesis, which is restrained by the cavity in which it is engaged, then remains in place, and the retention means 20, which have an inclined wall 33, easily leave the seat 22 of the prosthesis following the deformation of the tooth 16 in the release direction. By virtue of the control means 4, the opening movement of the attachment means can thus be easily controlled from outside the body of the patient by a surgeon.

After disconnection of the prosthesis and of the fitting and removing device, the latter can be withdrawn.

The removal of the prosthesis by the fitting and removing device can follow the same principle. The fitting and removing device, of which the attachment means are in the open position, is introduced into the supraglottal space until it reaches the prosthesis. The fitting and removing device is guided until the end 28 of the prosthesis enters the attachment seat 18, more precisely until the end 28 of the prosthesis comes into abutment against the limit stop 37. During the engagement of the attachment seat 18 around the end 28 of the prosthesis, the tooth 16 deforms in the release direction 17 when the protrusion 21 runs along the lateral wall of the end 28 of the prosthesis, until the protrusion 21 reaches the seat 22 of the prosthesis, in which seat 22 it engages. The cursor 10 is then moved in translation in the direction of the distal end 8 in such a way that the blocking means 23 are moved to the closed position. The blocking means 23 then extend along the full length of the tooth 16 in such a way as to prevent the deformations of the tooth 16 in the release direction. The prosthesis is then rigidly connected to the fitting and removing device. The assembly composed of the prosthesis and of the fitting and removing device is then withdrawn from the supraglottal space. During the removal operation, the fitting and removing device can be more easily guided by use of the guide wire 31.

Of course, the invention is not limited to the embodiments described with reference to the figures, and variations could be envisioned without departing from the scope of the invention. Thus, the movement of the attachment means from the open position to the closed position, and vice versa, under the control of the control means could be effected not by a movement of translation but instead by a movement of rotation or by a helical movement. Moreover, the attachment means could be inserted into the end 28 of the prosthesis, instead of the end 28 of the prosthesis being inserted into the attachment means. It is also possible to envision the use of other retention means and of other materials. Moreover, the fitting and removing device could permit the fitting and removal of other types of prostheses.

1. A device (1) for fitting and removing a prosthesis (40), said device (1) comprising:
attachment means (2) that can be attached to the prosthesis (40), the attachment means (2) being able to be arranged in two positions:
a closed position, in which they are rigidly connected to the prosthesis (40);
an open position, in which they are not rigidly connected to the prosthesis;
gripping means (3) for manipulation of the fitting and removing device (1);
control means (4), the control means (4) being connected to the attachment means (2), the control means (4) being able to control the attachment means (2) in such a way as to place same in the closed position or the open position;
at least partially flexible connecting means (5) that connect the control means (4) to the attachment means (2) in such a way as to transmit the command from the control means (4) to the attachment means (2).

2. The fitting and removing device (1) as claimed in claim 1, in which the attachment means (2) have:
an assembly zone (41) that can be joined to a part of the prosthesis (40);
an elastically deformable tooth (16) provided with retention means, (20) that are able to be retained by a complementary part (22) of the prosthesis (40), the tooth (16) having to deform elastically in at least one direction called the “release direction” (17) in order to allow the retention means (20) to free themselves from the complementary part (22) of the prosthesis (40);
blocking means (23) that can be placed in two positions:
a closed position, in which they prevent the deformation of the tooth (16) in the release direction (17);
an open position, in which they do not prevent the deformation of the tooth (16) in the release direction (17).

3. The fitting and removing device (1) as claimed in claim 2, in which the retention means (20) have a protrusion (21) that can be received in the complementary part (22) of the prosthesis (40).

4. The fitting and removing device (1) as claimed in claim 3, in which the blocking means (23) are formed by a ring (24).

5. The fitting and removing device (1) as claimed in one of claims 2 through 4, the fitting and removing device (1) extending along a reference axis (6), the blocking means (23) being able to move longitudinally so as to pass from the closed position to the open position, the control means (4) having a cursor (10) that can move longitudinally, the connecting means (5) having a connecting tube (29) which connects the cursor (10) to the blocking means (23) in such a way as to move the blocking means (23) longitudinally when the cursor (10) moves longitudinally.

6. The fitting and removing device (1) as claimed in one of the preceding claims, characterized in that it is additionally provided with guide means (15) in which a guide wire (31) can be inserted.

7. The fitting and removing device (1) as claimed in claim 6, in which the connecting tube (29) surrounds the guide means (15).

8. The fitting and removing device (1) as claimed in claim 7, in which the connecting means (5) and the guide means (15) form a flexible tube between the gripping means (3) and the attachment means (2).

9. The fitting and removing device (1) as claimed in claim 1, in which the attachment means (2) additionally have orientation means (25) that are able to orient the prosthesis (40) in rotation.

10. A kit for fitting and removing a prosthesis, said kit having:
a fitting and removing device (1) as claimed in claim 1;
a guide wire (31).

11. The fitting and removing device (1) as claimed in claim 1, for its use for fitting and/or removing a laryngeal or intralaryngeal prosthesis by the oral route.
